(19) United States
(12) Patent Application Publication
(10) Pub. No.: US 2024/0164349 A1
(43) Pub. Date: May 23, 2024

(54) HEALTH TRACKER DEVICE FOR HORSES

(71) Applicant: Tyler J. MORT, Ghent, NY (US)

(72) Inventor: Tyler J. MORT, Ghent, NY (US)

(21) Appl. No.: 18/548,547

(22) PCT Filed: Mar. 4, 2022
(86) PCT No.: PCT/US2022/018837

§ 371 Date: Aug. 31, 2023

Related U.S. Application Data
(60) Provisional application No. 63/156,826, filed on Mar. 4, 2021.

(51) Int. Cl. A01K 29/00; A01K 11/00; A61D 99/00; G16H 40/67

(52) U.S. Cl. CPC A01K 29/005; A01K 11/008; A61D 99/00; G16H 40/67

(57) ABSTRACT

A health tracker device for animals, including an electronics and hardware portion operatively attached to an expandable band, wherein the electronics and hardware portion includes a mechanism for detecting and tracking heart rate, a mechanism for detecting and tracking temperature, a mechanism for detecting and tracking position by GPS, a mechanism for detecting respiratory rate, a mechanism for detecting blood oxygen levels, a mechanism for detecting and differentiating movement (9-axis accelerometer), and a mechanism that vibrates to simulate a slow heart rate to calm an animal down. An application for monitoring health of an animal, and a method of using the application. A method of tracking the health of an animal, by securing the health tracker device to the tail of the animal, and detecting and tracking heart rate, temperature, GPS position, respiratory rate, blood oxygen levels, and movement of the animal.

### BACKGROUND OF THE INVENTION

### 1. Technical Field

The present invention relates to compositions and methods for tracking the health of animals. More specifically, the present invention relates to wearable health trackers for animals and especially horses.

### 2. Background Art

Horses undergo intense care throughout all their disciplines across the world. Whether it involves competition, breeding, recreation, or veterinary care, there will always be a need to acquire more health data on the animal to optimize training, care, or medical protocols. It can be advantageous to collect data on heart rate, temperature, and GPS position in monitoring horses and other animals.

Existing management of these parameters includes laborious vital collections by hand. A veterinarian is required to examine the animal with a thermometer and stethoscope. This can be inefficient and lacking in continuous health-data availability.

There are currently no devices created or manufactured in the United States that provide remote detection of health parameters for horses. In Switzerland, Piavita AG has created a girth strap that records heart rate, respiratory rate, temperature and movement. This device only works with BLUETOOTH®, so its communication range is severely limited. The custom girth design is bulky and inconvenient to horsemanship. The hair of the horse around the thorax extremely limits a strong sensory detection with the animal. Piavita claims the ability to predict birth in the horse. In France, a heart rate device (Equisense Motion S) with GPS exists that is built into a saddle with a custom girth strap and the device is set at the horse's heart. Use of this device is very expensive because it requires a custom saddle for each horse, and it cannot collect data when the horse is unsaddled and training. GPS devices have been placed on halters to monitor position, but these do not measure health parameters.

U.S. Patent Application 2017/0055496 to McHugh discloses a patch that can be attached to an animal's tail with adhesive, secured with a wrap, or strapped attachment. The patch is able to detect temperature with a sensor and transmit the temperature wirelessly to a server for a user to view data on a website or mobile device. The patch can also detect postural changes with an accelerometer and pulse with a pulse sensor. The patch can include GPS or triangulation-based location. The patch can be used to know when a horse is sick, when ready to give birth, and provide early signs of injury. While trainers can monitor the data, there is no disclosure that the patch can be used to enhance training by detecting heart rate. The '496 application also does not clearly disclose a sensor that is attached to an expandable band like a watch that is easy to remove.

WO 2017164807 to Andersson discloses a tail-mounted sensor for the health monitoring of animals such as horses, especially at the underside of the tail. The sensor detects heart rate and temperature. The sensor includes a fastening device attached to a housing. The housing of this device includes electronics and sits above the tail and appears bulky. A microprocessor can transmit sensed data wirelessly to an external receiver. The housing can also include a global navigation satellite system receiver to record position. There is no disclosure of use of the sensor in enhancing training.

Therefore, there remains a need for an easy-to-use health tracker for horses and other animals.

### SUMMARY OF THE INVENTION

The present invention provides for a health tracker device for animals, including an electronics and hardware portion operatively attached to an expandable band, wherein the electronics and hardware portion includes a mechanism for detecting and tracking heart rate, a mechanism for detecting and tracking temperature, a mechanism for detecting and tracking position by GPS, a mechanism for detecting respiratory rate, a mechanism for detecting blood oxygen levels, a mechanism for detecting and differentiating movement, and a mechanism that vibrates to simulate a slow heart rate to calm a horse down.

The present invention provides for an application for monitoring health of an animal, including a home screen, onboarding module, health tracker device module, map module, devices module, and stable module stored on non-transitory computer readable media.

The present invention provides for a method of using an application for monitoring the health of an animal, by a user onboarding with an onboarding module, the user logging in on a home screen, the user adding an animal profile in a stable module, the user adding a monitor in a health tracker device module, and the user viewing the location of animals being tracked with the map module.

The present invention provides for a method of tracking the health of an animal, by securing the health tracker device to the tail of the animal, wherein the health tracker device includes an electronics and hardware portion operatively attached to an expandable band, and detecting and tracking heart rate, respiratory rate, blood oxygen levels, accelerometer (movement), temperature, and GPS position of the animal.

The present invention provides for a method of tracking the health of an animal during training, by securing the health tracker device to the tail of the animal, wherein the health tracker device includes an electronics and hardware portion operatively attached to an expandable band, detecting and tracking heart rate, respiratory rate, blood oxygen, temperature, movement, GPS location, stride length, speed, balance/gait, force/impulsion/impact, and recovery/fitness data including heart rate return, heart rate variability, excess post-exercise oxygen consumption, and VO2 max during a training session, and adjusting the training based on data detected.

The present invention provides for a method of monitoring foals, by securing the health tracker device to the tail of a pregnant horse (wherein the health tracker device includes the electronics and hardware portion operatively attached to the expandable band), detecting and tracking heart rate, respiratory rate, pulse oxygen, temperature, and movement/activity with the health tracker device, and predicting birth of a foal in the horse.

The present invention provides for a method of calming an animal, by securing the health tracker device to the tail of the animal (wherein the health tracker device includes the electronics and hardware portion operatively attached to the expandable band), detecting and tracking heart rate, temperature, GPS position, respiratory rate, blood oxygen levels, and movement with the health tracker device, and providing vibration to the animal and simulating a low-normal heart rate to calm the animal.

### DESCRIPTION OF THE DRAWINGS

Other advantages of the present invention are readily appreciated as the same becomes better understood by reference to the following detailed description when considered in connection with the accompanying drawings wherein:

FIG. 1 is a side perspective view of the heath tracker device;

FIG. 2 is a side view of the health tracker device attached to a horse's tail;

FIG. 3 is a flowchart of screens in an application;

FIG. 4A is a diagram of information flowing between a health tracker device, a communications hub, a database, and an application, and FIG. 4B is a diagram of information flowing with multiple health tracker devices;

FIG. 5 is a diagram of communication between components of the device;

FIG. 6A is a front view of a housing and electronics and hardware portion, FIG. 6B is a bottom view of the housing and electronics and hardware portion, FIG. 6C is a lateral view of the housing and electronics and hardware portion, FIG. 6D is an oblique view of the housing and electronics and hardware portion, FIG. 6E is an oblique view of the housing and electronics and hardware portion, and FIG. 6F is top view of the housing and electronics and hardware portion;

FIG. 7A is a screenshot of a home screen in an application, FIG. 7B is a screenshot of a monitor view in the application, FIG. 7C is a screenshot of a new monitor in the application, FIG. 7D is a screenshot of a map module in the application, FIG. 7E is a screenshot of a device module in the application, FIG. 7F is a screenshot of connecting new devices in the application, and FIG. 7G is a screenshot of adding a new monitor, horse, or watch;

FIG. 8A is a side perspective view of the health tracker device, and FIG. 8B is a transparent side perspective view of the health tracker device showing an antenna; and

FIG. 9A is a front view of a health tracker device with an over band, FIG. 9B is a top perspective view of a health tracker device with an over band, FIG. 9C is a side perspective view of a health tracker device with an over band, and FIG. 9D is a bottom view of a health tracker device with an over band.

### DETAILED DESCRIPTION OF THE INVENTION

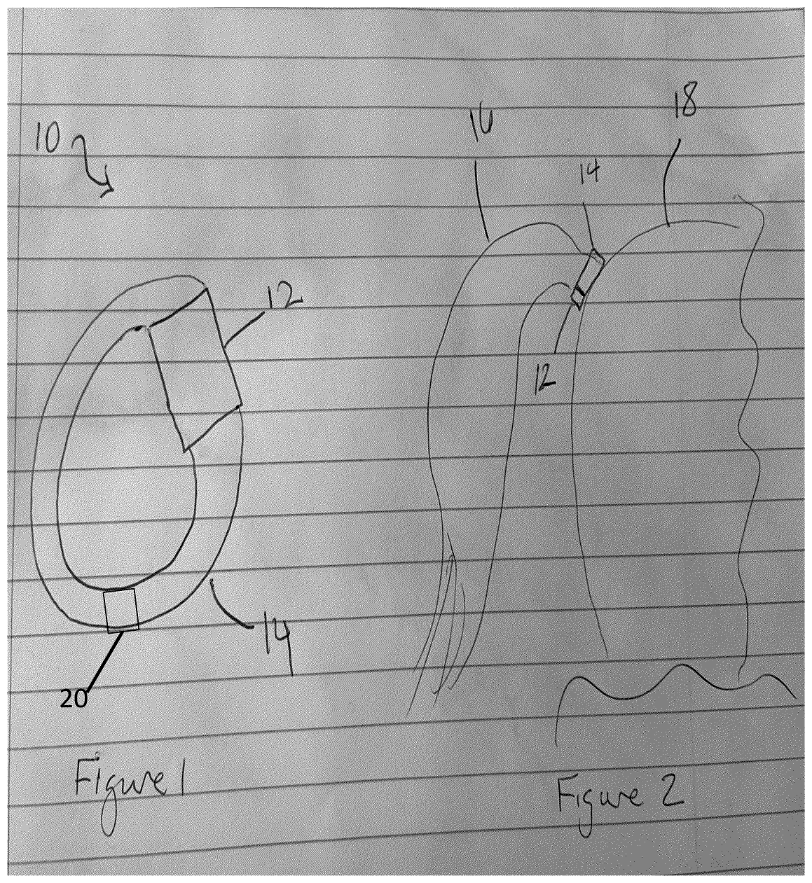

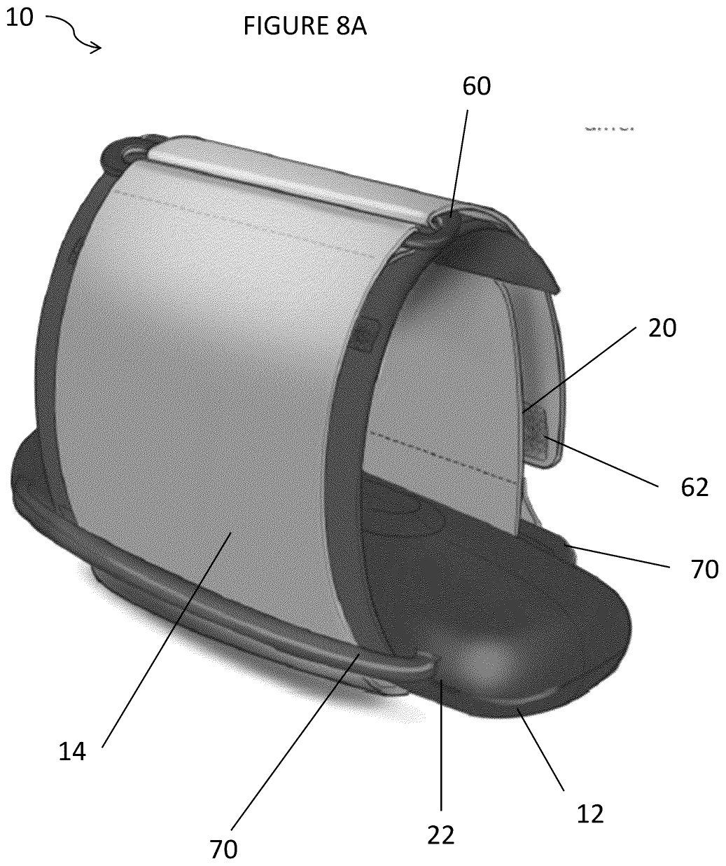

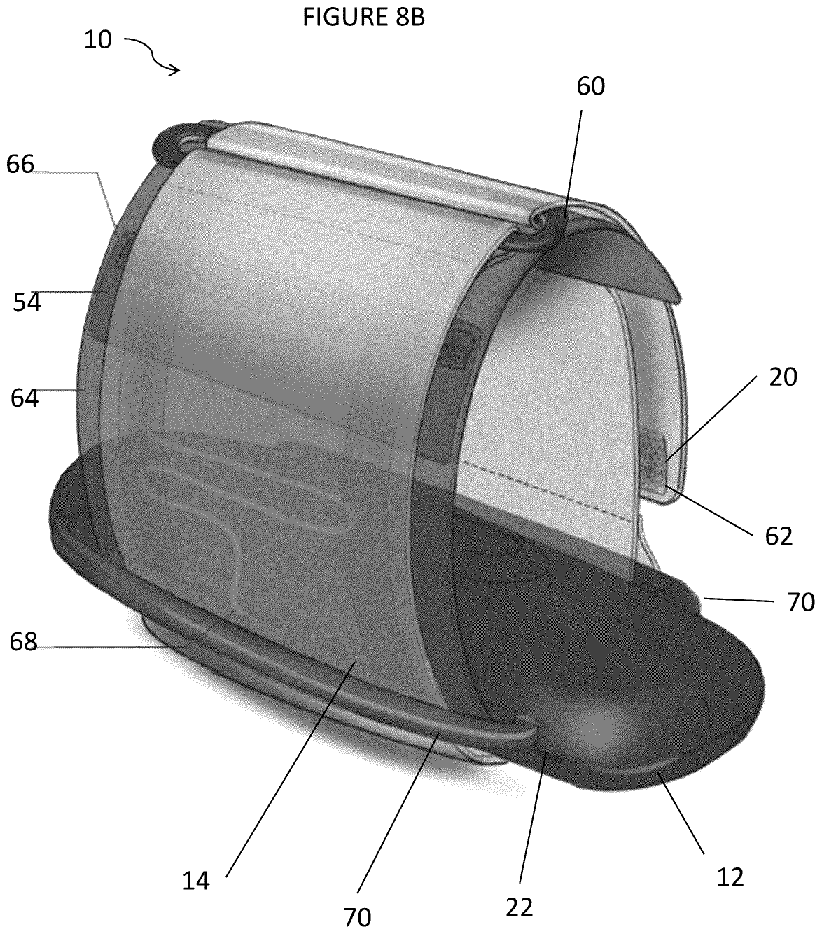

The present invention provides for a health tracker device 10 for horses and other animals 18 that can detect and track heart rate, temperature, position by GPS, respiratory rate, blood oxygen levels, movement, and provide vibration to simulate a slow heart rate to calm an animal down, including an electronics and hardware portion 12 operatively attached to an expandable band 14. The health tracker device 10 is generally shown in FIGS. 1, 8A, and 8B.

“Animal” as used herein refers to non-human mammals, and can include horses, cows, sheep, pigs, dogs, cats, giraffes, rhinoceroses, camels, donkeys, alpacas, buffalo, goats, and others.

In general, the health tracker device 10 is shaped like a watch wherein the electronics and hardware portion 12 in a housing 22 can be secured to an animal's tail 16 with a tough, flexible, and expandable band 14, shown in FIG. 2.

The housing 22 can be any suitable hard plastic such as acrylic, polycarbonate, polyethylene, polypropylene, polyethylene terephthalate, polyvinyl chloride, or acrylonitrile-butadiene-styrene. The housing 22 can be any suitable size to enclose all necessary hardware and devices in the electronics and hardware portion 12, such as 21.5 mm in height, 40 mm in width, and 77.9 mm in length. The housing 22 can include at least one band receiving slot 70 to receive the band 14 and secure the band 14 to the housing 22.

The band 14 can be made of soft, strong, stretchable material such as, but not limited to, neoprene, elastane (LYCRA®, the Lycra Company), nylon, polypropylene, or others. The band 14 is designed to stay in place on the animal. It needs to be tight enough to stay on the animal, because they swish their tails and rub on things, but it also needs to be “gentle” or “loose” enough that it does not too aggressively compress the device 10 against the skin that can be sensitive and cause sores when it is used for more than 24-48 hours at a time, while still promoting enough contact to ensure proper sensor readings. The band 14 allows several things. The device 10 is tightly secured so that it does not come off. The band 14 does not compress the device 10 too aggressively against the skin, while still allowing for enough contact with skin and blood vessels to ensure clear readings. The device 10 must be placed the furthest “cranially” on the tail (as close to the body as possible), as the tail is strongest and thickest here. This allows for tightening the band 14 maximally and there is a larger surface area of skin and blood vessels available here. The device 10 is not intended to be used anywhere else on the body as this location is the only one that has bare skin, accessible blood vessels, and a convenient, cylindrical structure like the tail to band 14 itself to. The band 14 can include a securing mechanism 20 such as a buckle 60 and/or hook and loop 62 to provide a tight and snug fit against the tail 16. The band 14 can have a portion that threads through a buckle 60 and folds back to secure itself with hook and loops 62, shown in FIG. 8A. The band 14 can be sized to fit different animals. For example, the band 14 can be sized to fit a circumference of 150-300 mm. The band 14 can be machine-washed and it protects the components of the device 10 itself from being contaminated by pathogens of a possibly sick animal.

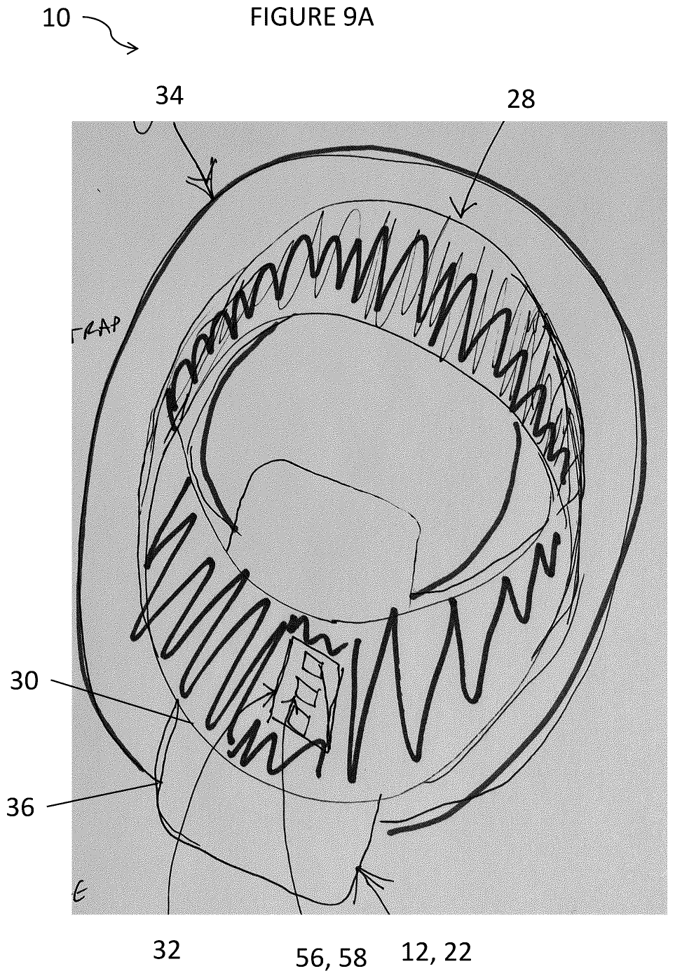

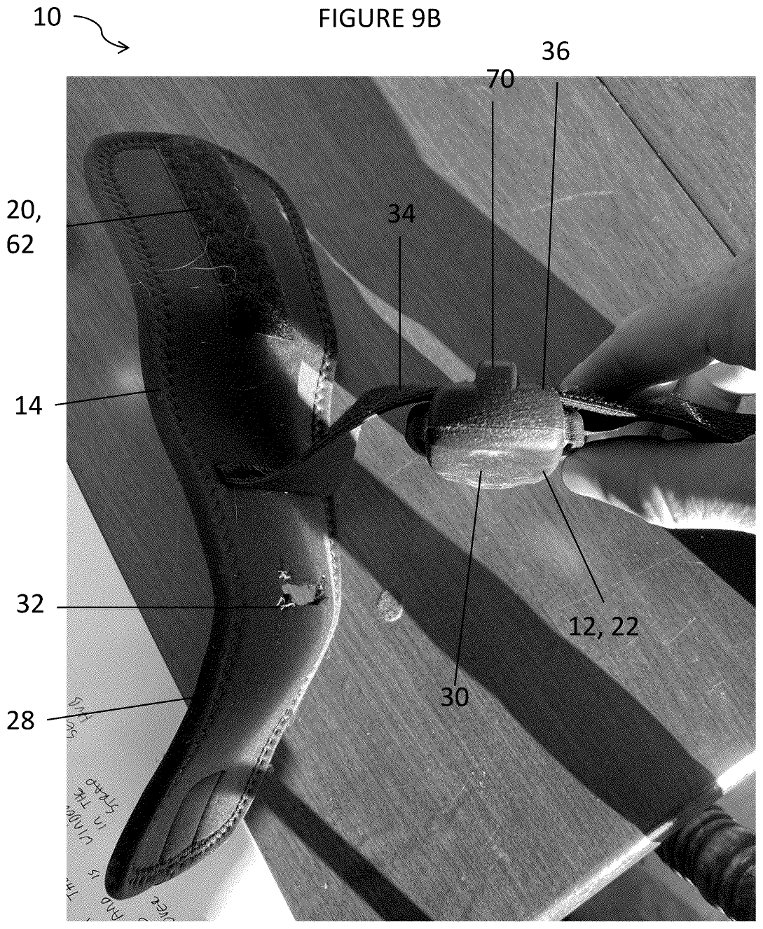

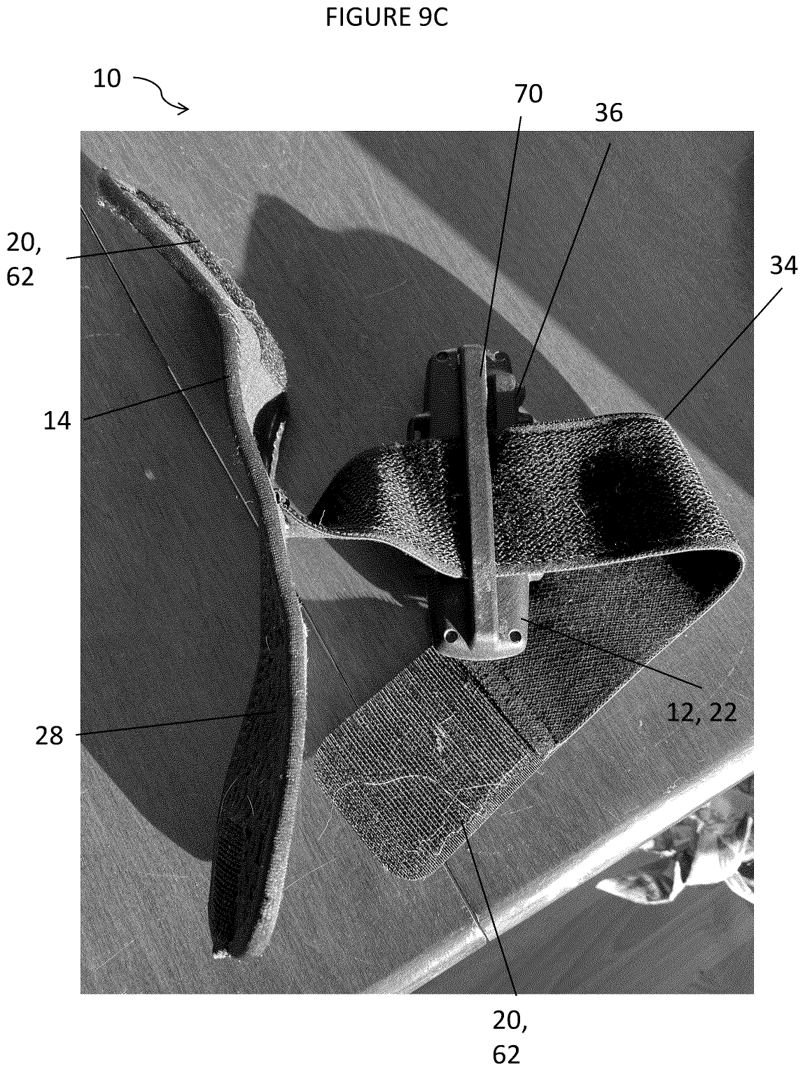

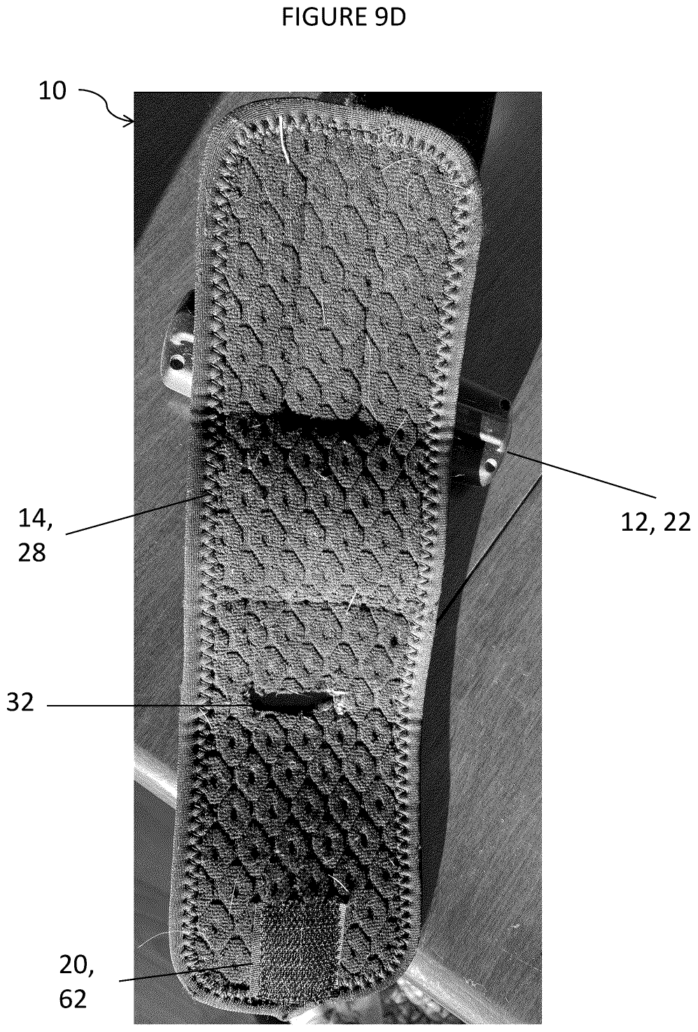

The band 14 can be a single piece or include multiple pieces such as shown in FIGS. 9A-9D. An under band 28 can directly contact the tail 16 and faces a bottom side 30 of the housing 22, and includes an opening 32 through which any necessary sensors (temperature sensor 56, LED sensor hub 58) on the electronics and hardware portion 12 can contact the tail 16. The securing mechanism 20 secures the under band 28 to the tail 16 for a snug fit, and can be located at any suitable location on the under band 28. An over band 34 is operably attached to the under band 28 at any suitable location and faces a top side 36 of the housing 22. The over band 34 wraps around the under band 28 and on top of the housing 22 through a band receiving slot 70 and is secured with a second securing mechanism 20. The over band 34 supports an antenna 54 such that the antenna 54 is positioned to wrap and conform to the sides of the tail 16 and top side of the tail 16, covering transmission angles between minus 170 degrees and positive 170 degrees from tail top center vertical. The antenna 54 is flexibly encased and outwardly facing for wireless electronic communication, further described below. The antenna 54 is dimensionally configured to wrap substantially around the animal's tail 16 to optimize transmission direction according to the diameter of the animal's tail 16.

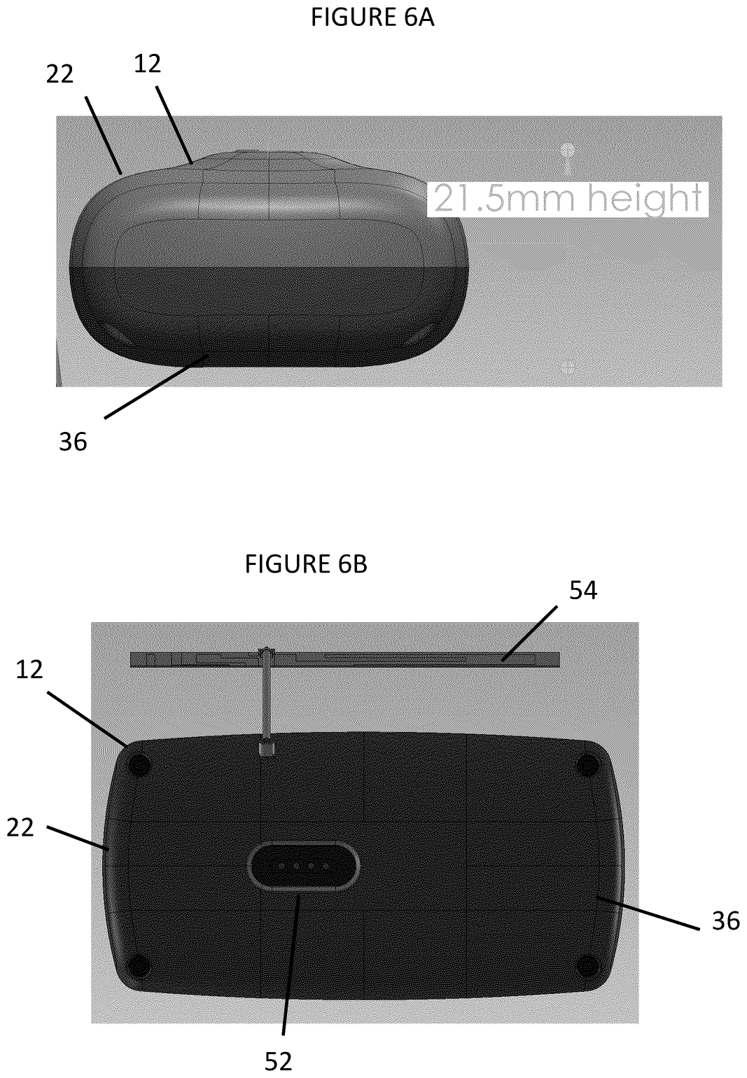

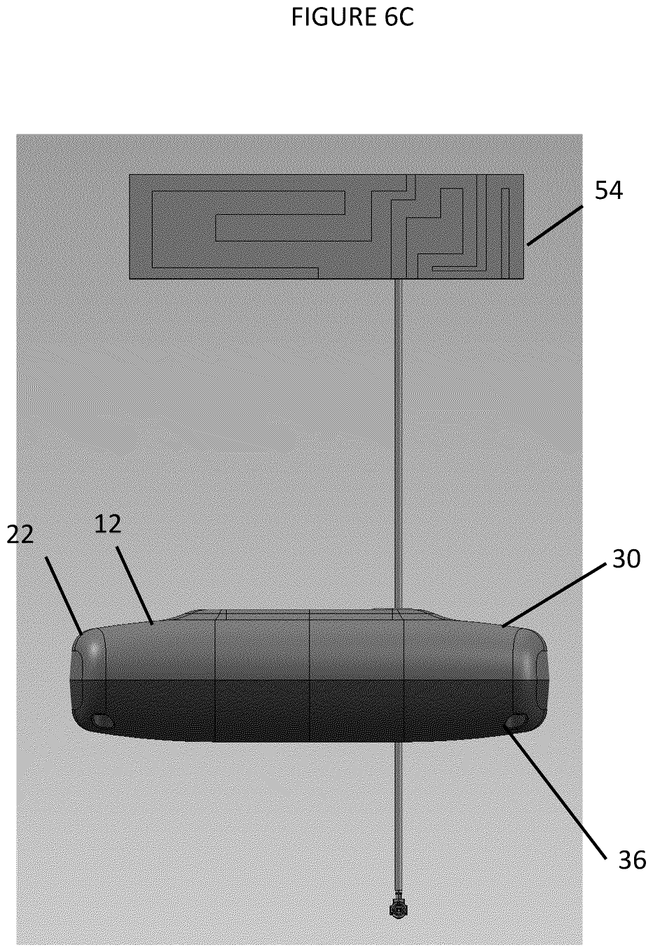

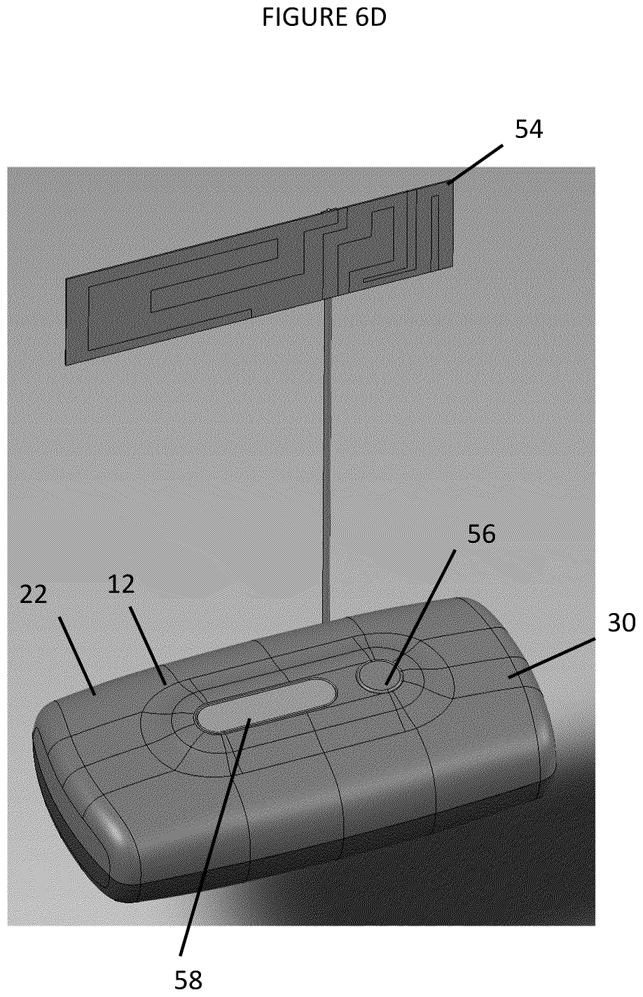

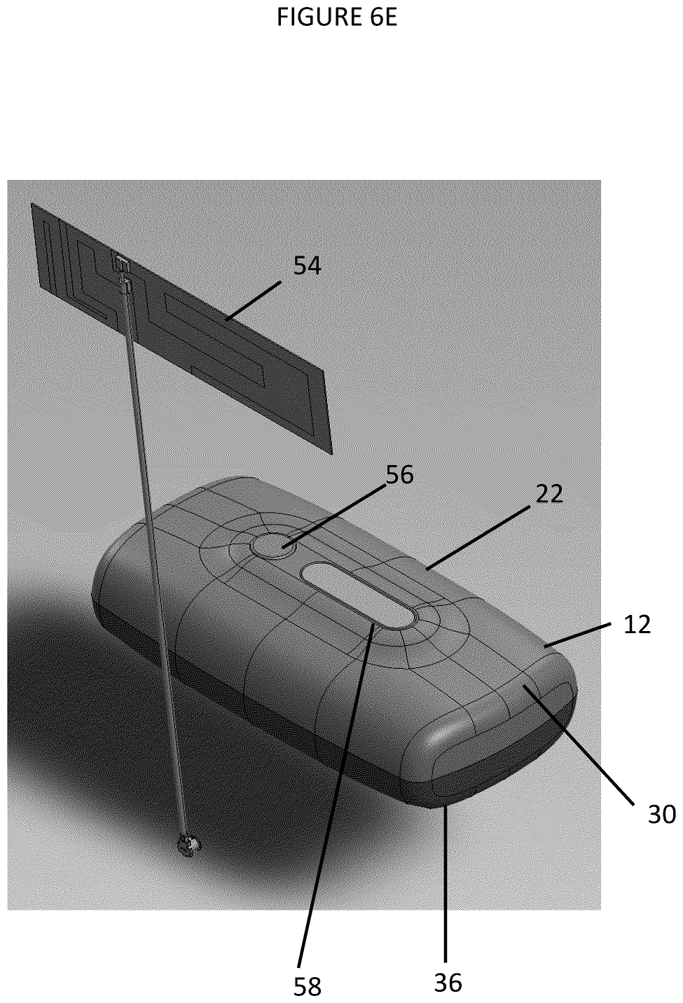

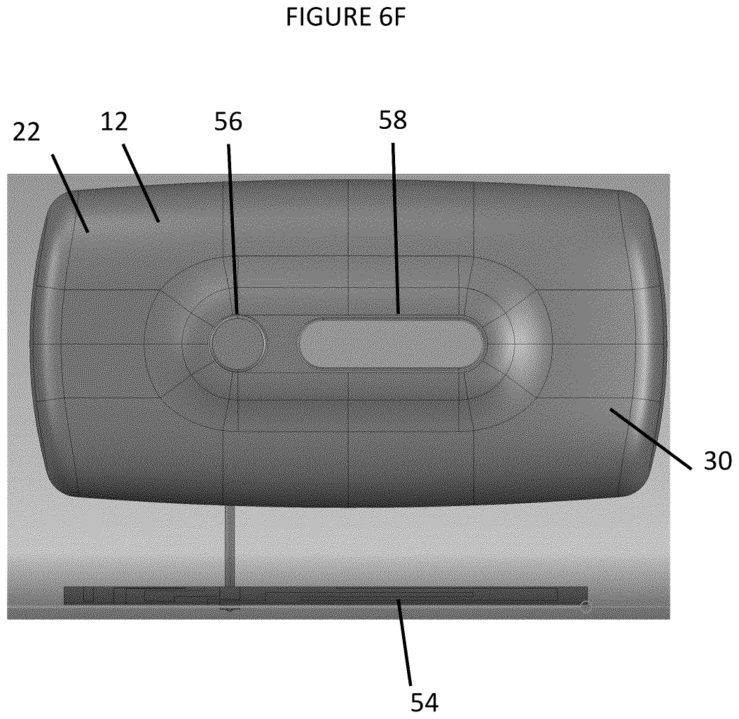

FIGS. 6A-6F show the housing 22 enclosing the electronics portion 12. The electronics and hardware portion 12 includes any necessary electronics to measure the parameters desired (i.e., mechanisms for detecting and tracking each parameter, sensors). For example, heart rate can be measured via an LED light (the LED can see through the animal's skin to measure blood flowing through veins) or an electrode. Temperature can be measured via a metal probe, infrared light, or silicon temperature sensor mounted to a stainless-steel component 56 in direct skin contact. GPS tracking is performed via a micro-chip. Respiratory rate can be detected with sensors (such as inductance plethysmography sensors, capnography sensors, piezoelectric sensors, bioimpedance-based sensors, acoustic sensors, optical sensors). Respiratory rate can be detected with a reading of the detected heart rate, more specifically, the change in Heart Rate Variability (HRV). The length of time between consecutive heartbeats shortens slightly as the horse inhales and lengthens as it exhales. The term for this biological phenomenon is respiratory sinus arrhythmia (RSA). Blood oxygen levels can be detected with sensors (such as SPO2 pulse oximeters). Movement can be detected and differentiated with a 9-axis accelerometer/gyroscope/magnetometer which can be a combination chip. Any sensors using LED can be grouped in a sensor hub 58. Vibration can be provided to simulate a slow heart rate to calm an animal down with any vibration mechanism such as a vibration motor capable of simulating heartbeat patterns. An LED sensor can be included for glucose monitoring that can read blood glucose concentrations, such as for nutritional monitoring, metabolic pathway monitoring (such as for equine metabolic syndrome), and physiological pathology monitoring (such as for laminitis). A blood pressure mechanism can be included that allows the band 14 to expand very slightly with air to take blood pressure of the animal, such as for veterinary use. Electrolyte and hydration levels can be monitored with an LED sensor that can read changes in hydration states and general electrolyte concentrations. This is useful for monitoring sick animals, especially with diarrhea or fluid administration management. It is also useful for horses in training, especially with common issues such as Rhabdomyolisis, aka “Tying-up”. It is also useful for animals that live in dry areas that are prone to impactions of their digestive system from over consumption of sand.

Measurements can be taken continuously, or at set times, such as once every 5 minutes, 10 minutes, 15 minutes, 20 minutes, etc. All data obtained by these mechanisms and sensors can be stored by the health tracker device 10 and/or sent electronically by BLUETOOTH® or LORAWAN® to an application 26 or communication hub 24 further described below. Preferably, up to 8 hours of data can be stored on the health tracker device 10, and once the data is transferred to the application 26 successfully, the health tracker device 10 receives an acknowledgement receipt and clears the memory in the storage.

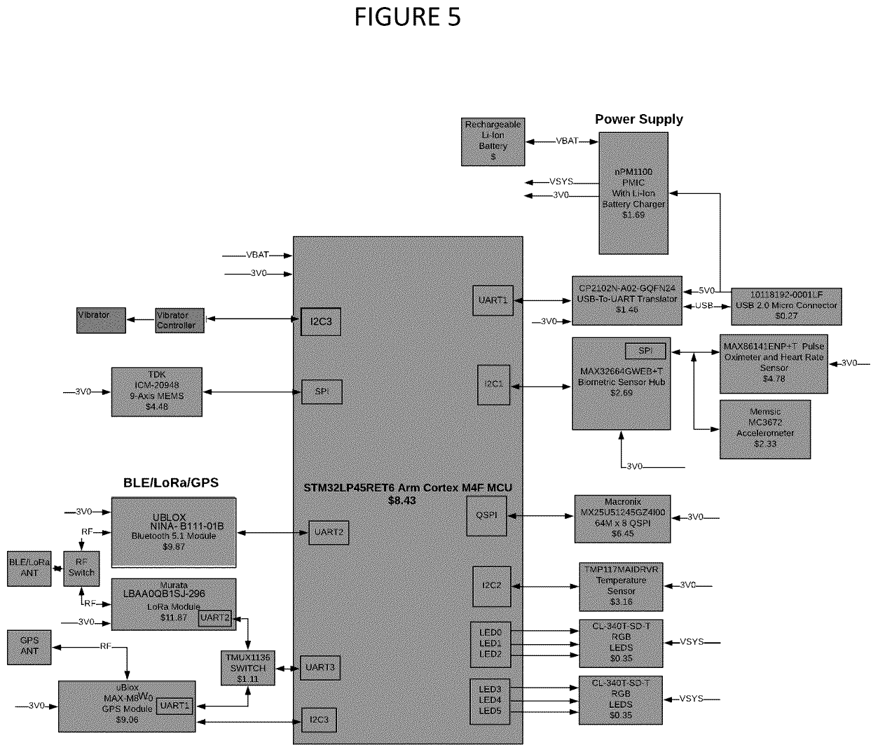

The electronics and hardware portion 12 can also include BLUETOOTH® or LORAWAN® communication mechanisms including at least one antenna 54 for connecting with mobile devices. The antenna 54 can be created by printed undulating traces configured to optimize transmission strength and conformity according to the diameter of the animal's tail 16. The antenna 54 can feed into the band 14 to prevent an animal's body tissue from blocking signals. The antenna 54 can click into a button on the outside of the housing 22 to help water resistance. The band 14 can include a pocket 64 made of fabric to secure and house the antenna 54, and hook and loop antenna securing mechanisms 66 inside the pocket 64, shown in FIG. 8B. The antenna 54 can include a cord 68 that connects to electronics (PCBA) in the electronics and hardware portion 12 within the housing 22. The band 14 can contain a flexible portion of a rigid-flex PCBA where an inner layer of a flexible circuit portion includes a reflective layer below the antenna traces. The electronics and hardware portion 12 can be optionally in electronic communication with a screen for displaying parameters. The electronics and hardware portion 12 can include a battery (replaceable or rechargeable such as a 500 mAh 3.7v LiPo battery), an integrated protection circuit module (PCM) to prevent the battery from overcharging or over discharging, a thermistor to monitor battery and electronics temperature for safe operation, and any computer hardware (printed circuit board assembly (PCBA)), computer storage, or computer readable media necessary to run the device and collect and store data. The battery can be charged by magnetic USB port 52 with a mobile battery pack while attached to an animal so that the health tracker device 10 does not need to be removed, and the mobile battery pack can fit in a pocket on the band 14. Other ports can be included, such as micro USB or Type C for charging the battery or for data transfer. Preferably, the battery can last over a month under continuous use when in a long-term mode. The electronics and hardware portion 12 can include a user interface including a power button (that can toggle between on and off), reset button that can be used to reset the device, at least one multi-function RGB LED, and a 4-pin magnetic charging and data transfer connection. The RGB LED can use multiple colors to indicate various device status indications such as red, green, blue, yellow, and white, and can use pulsed (sinusoidal brightness variation), blinked, or steady. For example, green pulsing or continuous can be the default state when the device 10 is on, red steady can indicate that the battery is charging, yellow steady can indicate the battery charge is low, blue pulsing can indicate attempting to establish wireless connection to the application 26, and blue stead can indicate connection to the application 26. FIG. 5 shows a diagram of communication between processes and parts in the electronics and hardware portion 12.

The health tracker device 10 is most preferably placed around the base of the tail 16 of the horse or animal 18, where it continually reads heart rate, body temperature, GPS position information, respiratory rate, blood oxygen levels, and movement for the animal 18. As described above, the electronics and hardware portion 12 is facing against the ventral face of the tail 16 for proper data collection, due to the lack of hair on this portion of the tail 16. It is desired for the device to be contact with the animal's skin in an area where it is not easy to rub or scratch the health tracker device 10 off the animal. In a horse, one of the few places on the body with open skin is at the base of the tail, and a vein runs through this area that allows for easier measurement of heart rate. The ankle is another area on a horse that the health tracker device 10 can be placed, but this area is also prone to being scratched or covered in mud by a horse. A veterinarian can also shave off an area of hair on an animal to access skin for placement of the health tracker device 10 if needed. The health tracker device 10 preferably weighs less than 200 g. The health tracker device 10 can withstand moderate vibration from a galloping animal.

The health tracker device 10 is in electronic communication via BLUETOOTH® or LORAWAN® (LoRa Alliance) to an application 26 stored on a user's mobile device (i.e., smartphone or tablet computer) on non-transitory computer readable media. LORAWAN® can send data up to 28 miles away and is a low bandwidth, low data rate technology. BLUETOOTH® does not transmit through body tissue, so it can be extremely unreliable with horses or other animals that have a lot of body tissue so LORAWAN® provides another option for data transmission. All data tracked and collected by the health tracker device 10 can be sent to the application 26. The data can help in providing efficient training of animals, because by monitoring heart rate, one can see if the training is too easy for animal and they can be pushed harder to increase their heart rate. The data can also help veterinarians in monitoring fever (temperature) and heart rate in animals with severe illnesses.

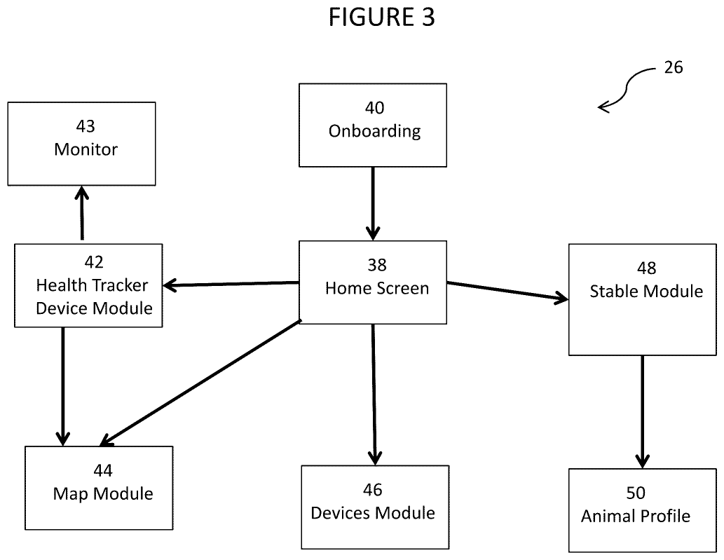

The application 26 is further shown in FIG. 3 and includes a home screen 38, onboarding module 40, health tracker device module 42, map module 44, devices module 46, and stable module 48.

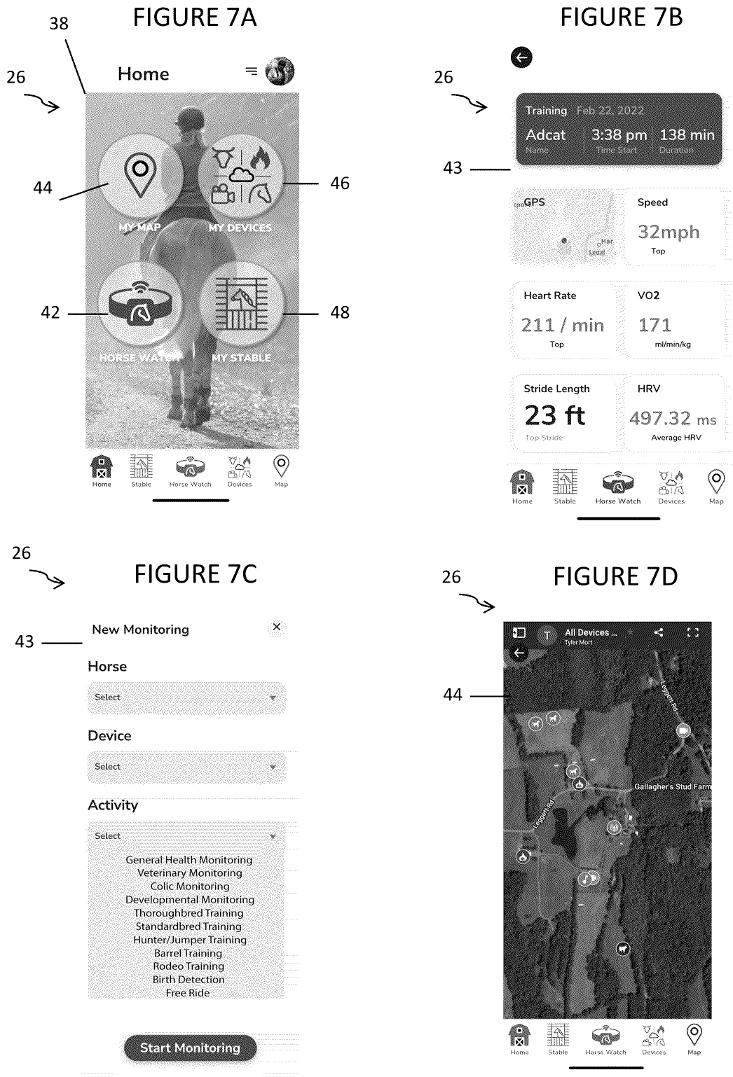

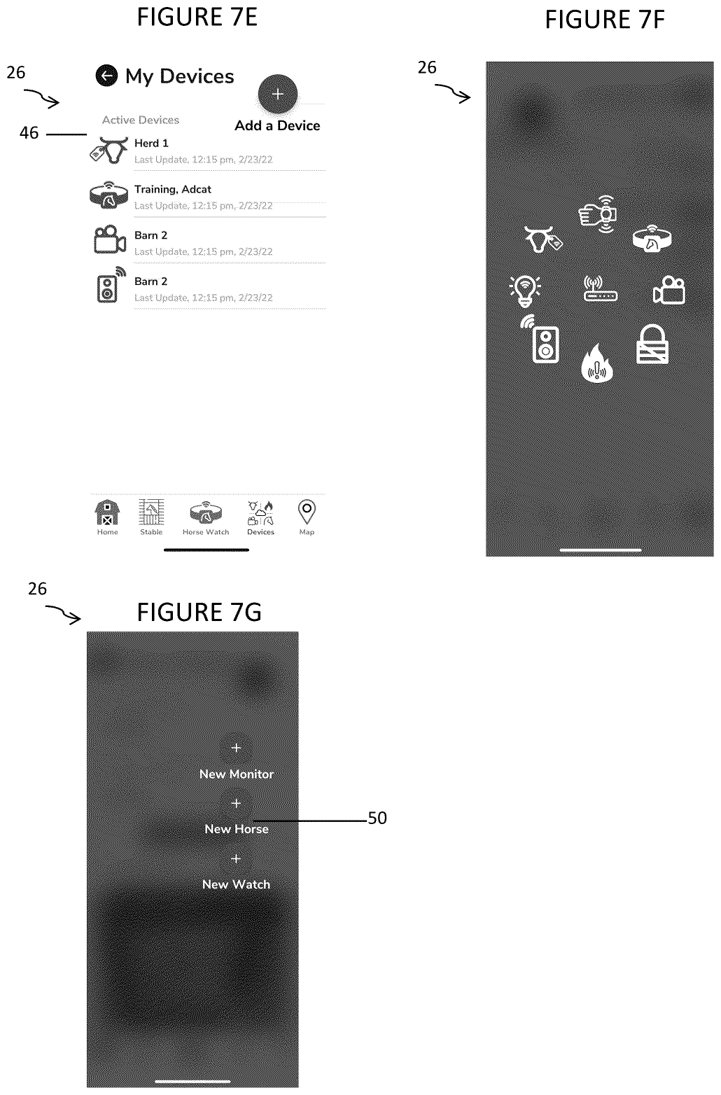

Home screen 38, shown in FIG. 7A, allows for a user to login with a username and password, and provides access to an onboarding module 40, health tracker device (horse watch) module 42, map module 44, devices module 46, and stable module 48.

Onboarding module 40 allows the user to sign up on the application 26 and enter personal details such as creating a username and password.

The health tracker device module 42 shows a list of monitors 43 (such as active monitors, recent monitors, and all monitors) and links to a monitoring map in the map module 44. By accessing all monitors, the user has a list of all monitors 43. Selecting a specific monitor 43 shows details of the monitor 43, shown in FIG. 7B, such as for a training session, start time and duration are shown along with top speed, top heart rate, VO2, top stride length, average HRV, EPOC, and HRR at 10 minutes. A new monitor 43 can also be added, shown in FIG. 7C.

The map module 44 allows the user to zoom in and out to view where animals being tracked or connected devices are located. FIG. 7D shows a map module 44.

The devices module 46, shown in FIG. 7E, shows a list of all devices connected with the application 26, such as a bull monitor, smoke detectors, farm cameras, and one or multiple health tracker devices 10. Each device can be selected to view further details. New devices can also be connected to the application 26, shown in FIG. 7F.

The stable module 48 shows animals being tracked and allows a user to add an animal profile 50 to the stable (FIG. 7G, add new horse). The animal profile 50 provides details about the animal (breed, age, sex, height, weight, owner, veterinarian), monitorings, and notes.

A method of using the application 26 is provided, by a user onboarding with the onboarding module 40, a user logging in on the home screen 38, a user adding an animal profile 50 in the stable module 48, the user adding a monitor 43 in the health tracker device module, the user viewing the location of animals being tracked with the map module 44, and optionally the user viewing devices linked with the application in the devices module 46.

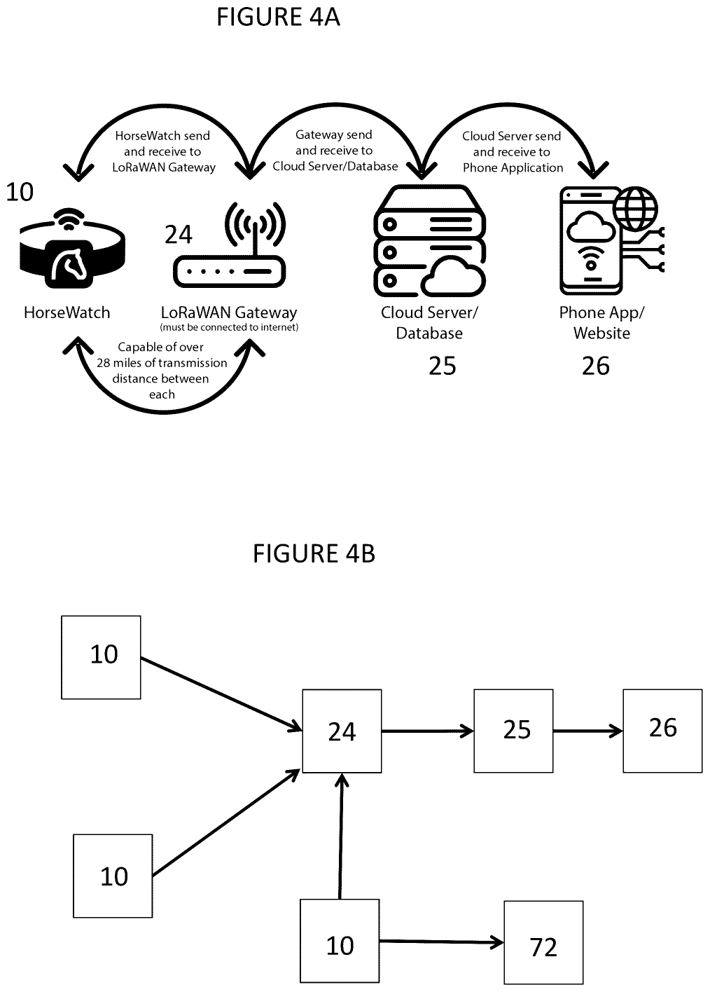

While the health tracker device 10 can communication with the application 26 directly, it can also be in electronic communication with a communications hub 24 via LORAWAN®, shown in FIGS. 4A-4B. Data can be sent from the communications hub 24 to a database or cloud server 25 and then to the application 26 on a mobile device. Multiple health tracker devices 10 can be in communication with the communications hub 24 as shown in FIG. 4B, as well as other devices such as, but not limited to, a herd bull tracker, barn cameras, smoke detectors (such as those in barns), RFID crosshairs, and smart gates. The communications hub 24 can communicate electronically with the application 26 so that any devices can be managed in the devices module 46.

The health tracker device 10 can also be in electronic communication via BLUETOOTH® with a rider mobile device 72, such as a smart watch. The rider mobile device 72 can display in clear and big letters any pertinent information or parameters that the rider might want to know and view while riding such as speed, heart rate, stride length, jumping force, or any other parameters desired. This can be useful for the rider to know if they need to make adjustments in a training session.

The health tracker device 10 can remain on the animal if tracking is needed, such as for a training period to monitor training, or for longer periods to monitor illness in an animal, or to monitor the general health in an animal. The health tracker device 10 can remain on the animal for hours, days, weeks, or months. For example, the health tracker device 10 can be placed on several horses during a training period, and then taken off and put on other horses during a second training period. The health tracker device 10 can be always kept on to monitor animals when they are grazing in pastures.

The present invention provides generally for a method of tracking the health of an animal 18, by securing the health tracker device 10 to the tail 16 of the animal 18, and detecting and tracking heart rate, temperature, GPS position, respiratory rate, blood oxygen levels, and movement. The method can further include sending tracked data to the application 26 on a mobile device and/or the communications hub 24. The method can further include providing vibration to the animal to simulate a slow heart rate to calm the animal down. The method can further include any of the steps further detailed below for specific uses.

There are several uses for the health tracker device 10. The health tracker device 10 can be used to monitor health, development, and training. Various parameters can be monitored, such as, but not limited to, speed, top speed during the week, top acceleration, average daytime heart rate, and stride length.

For general health monitoring, the health tracker device 10 can be used in veterinarian clinics, farms, training facilities, or anywhere that an animal is at. There can be several reasons for general health monitoring, such as the horse is showing signs of illness, the owner wants to monitor general wellbeing, monitoring young animals to determine their abilities, to evaluate health, nutrition, and fitness, or the owner would like to track their location. For this purpose, heart rate, respiratory rate, pulse oximetry, temperature, movement/activity, and GPS location can be monitored. Averages of these data points can be sent out to the application 26 and/or communications hub 24 approximately every 5 minutes.

For training, the health tracker device 10 can be used in training centers, farms, ranches, or anywhere that the animal is at. Use in training allows for quantifying training metrics that have not previously been quantified, and analyzing all aspects of horse sports. Every sport is different and values different things such as speed, strength, gait, jumping, working cattle, etc. For this purpose, heart rate, respiratory rate, blood oxygen, temperature, movement, GPS location, stride length (by using a combination of GPS location and the accelerometer), speed (using GPS location over time), balance/gait (using the accelerometer which can determine lameness through statistical deviations), force/impulsion/impact (using the accelerometer), and recovery/fitness data including heart rate return (how quickly the heart rate returns to baseline), heart rate variability (the change in time between each heart beat), excess post-exercise oxygen consumption (the oxygen debts that an animal has to recover after a workout), and VO2 max (the maximum amount of oxygen that an animal can utilize during intense or maximal exercise, generally considered the best indicator of cardiovascular fitness and aerobic endurance). Data can be sent to the application 26 and/or communications hub 24 once per second during training, and between once every 5 to 10 minutes during a resting period.

Therefore, the present invention provides for a method of tracking the health of an animal 18 during training, by securing the health tracker device 10 to the tail 16 of the animal 18 (wherein the health tracker device 10 includes the electronics and hardware portion 12 operatively attached to the expandable band 14), detecting and tracking heart rate, respiratory rate, blood oxygen, temperature, movement, GPS location, stride length, speed, balance/gait, force/impulsion/impact, and recovery/fitness data including heart rate return, heart rate variability, excess post-exercise oxygen consumption, and VO2 max during a training session, and adjusting the training based on data detected.

Use with training can be used for all breeds of horses and disciplines, such as, but not limited to, Quarter Horses (rodeo, barrels, roping), thoroughbred (racing to determine speed, fitness, stride length, tracking, and recovery), hunter/jumper (to determine impulsion when jumping, gait monitoring, and stride length), polo (to determine fitness and speeds), endurance (to determine fitness, physiological status, speeds, and GPS location), standardbreds (same a thoroughbred parameters), paso finos (to determine gait, balance, and fitness), and draft horses (to determine force, impulse, and fitness).

The health tracker device 10 can provide a performance analysis, including a lameness indicator based on accelerometer mapping, or a recovery analysis. In ill animals, the health tracker device 10 can be used to monitor heart rate and temperature to monitor progress of disease state.

The health tracker device 10 can be used to monitor foals, and determine when a horse is expected to foal (birth prediction). This can be used in breeding farms, veterinary clinics, or anywhere with a pregnant horse. Horse births are extremely unpredictable and have a high rate of catastrophe for mother and baby, and therefore there is a need to monitor pregnant horses. The health tracker device 10 can monitor heart rate, respiratory rate, pulse oxygen, temperature, and movement/activity. By detecting a unique change in all of these parameters, birth can be predicted. The health tracking device 10 can send data to the application 26 and/or communications hub 24 every 10 minutes, and can be used long term for this purpose (weeks to a month).

Therefore, the present invention provides for a method of monitoring foals, by securing the health tracker device 10 to the tail 16 of a pregnant horse (wherein the health tracker device 10 includes the electronics and hardware portion 12 operatively attached to the expandable band 14), detecting and tracking heart rate, respiratory rate, pulse oxygen, temperature, and movement/activity with the health tracker device 10, and predicting birth of a foal in the horse.

The health tracker device 10 can be used to calm an animal wherein the device 10 vibrates, simulating a low-normal heart rate (around 35 bpm). This can be indicated for horses with anxiety due to disturbances such as missing their friend, travelling, wanting to go outside, etc. This can also be indicated for horses with behavioral issues like “cribbing”, “weaving”, “Stall walking” or any other issues. Horses find the vibration therapeutic. This can be used anywhere with a horse, veterinary clinics, training farms, breeding farms, small farms, or horse shipping companies. The health tracker device 10 can be placed on the horse for as long as the calming therapy is desired or needed.

Therefore, the present invention provides for a method of calming an animal 18, by securing the health tracker device 10 to the tail 16 of the animal 18 (wherein the health tracker device 10 includes the electronics and hardware portion 12 operatively attached to the expandable band 14), detecting and tracking heart rate, temperature, GPS position, respiratory rate, blood oxygen levels, and movement with the health tracker device 10, and providing vibration to the animal 18 and simulating a low-normal heart rate to calm the animal 18.

The health tracker device 10 can be used in the competitive industry, veterinary health management, general horse farming, or general horse recreation.

Throughout this application, various publications, including United States patents, are referenced by author and year and patents by number. Full citations for the publications are listed below. The disclosures of these publications and patents in their entireties are hereby incorporated by reference into this application to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible considering the above teachings. It is, therefore, to be understood that within the scope of the appended claims, the invention can be practiced otherwise than as specifically described.

What is claimed is:

1. A health tracker device for animals, comprising an electronics and hardware portion encased in a housing operatively attached to an expandable band, wherein said electronics and hardware portion includes a mechanism for detecting and tracking heart rate, a mechanism for detecting and tracking temperature, a mechanism for detecting and tracking position by GPS, a mechanism for detecting respiratory rate, a mechanism for detecting blood oxygen levels, a mechanism for detecting and differentiating movement (9-axis accelerometer), and a mechanism that vibrates to simulate a slow heart rate to calm an animal down.

2. The health tracker device of claim 1, wherein said band is made of stretchable material chosen from the group consisting of neoprene, elastane, nylon, and polypropylene.

3. The health tracker device of claim 1, wherein said band includes a securing mechanism chosen from the group consisting of a buckle and hook and loop.

4. The health tracker device of claim 1, wherein said band includes an under band for directly contacting an animals tail facing a bottom side of said housing, said under band including an opening through which said electronics and hardware portion contacts the tail and a securing mechanism, and an over band operably attached to said under band facing a top side of said housing, said over band wrapping around said under band and on top of said housing and including a second securing mechanism.

5. The health tracker device of claim 1, wherein said mechanism for detecting and tracking heart rate is chosen from the group consisting of an LED light and an electrode.

6. The health tracker device of claim 1, wherein said mechanism for detecting and tracking temperature is chosen from the group consisting of a metal probe, infrared light, and silicon temperature sensor.

7. The health tracker device of claim 1, wherein said mechanism for detecting and tracking position by GPS includes a micro-chip.

8. The health tracker device of claim 1, wherein said mechanism for detecting respiratory rate is further defined as a sensor that detects change in heart rate variability.

9. The health tracker device of claim 1, wherein said mechanism for detecting blood oxygen levels is further defined as an SPO2 pulse oximeter.

10. The health tracker device of claim 1, wherein said mechanism for detecting and differentiating movement is further defined as a 9-axis accelerometer, gyroscope, and magnetometer.

11. The health tracker device of claim 1, wherein said mechanism that vibrates to simulate a slow heart rate to calm an animal down is further defined as a vibration motor.

12. The health tracker device of claim 1, wherein said electronics and hardware portion further includes BLUETOOTH® and LORAWAN® communication.

13. The health tracker device of claim 1, wherein said electronics and hardware portion further includes antennas, a battery, a thermistor, computer hardware, and computer storage.

14. The health tracker device of claim 1, wherein said electronics and hardware portion further includes a user interface having a power button, reset button, at least one multi-function RGB LED, and a 4-pin magnetic charging and data transfer connection.

15. The health tracker device of claim 1, wherein said electronics and hardware portion is in electronic communication with a screen for displaying parameters.

16. The health tracker device of claim 1, wherein said health tracker continually detects and tracks heart rate, temperature, position, respiratory rate, blood oxygen levels, and movement.

17. The health tracker device of claim 1, wherein said electronics and hardware portion further includes a mechanism for monitoring glucose, a mechanism for monitoring blood pressure, and a mechanism for monitoring electrolyte and hydration.

18. The health tracker device of claim 1, further in electronic communication with a rider mobile device for viewing parameters detected while riding.

19. The health tracker device of claim 13, wherein said antenna is further defined as a flexibly encased outwardly facing antenna for wireless electronic communication encased in said band.

20. The health tracker device of claim 19, wherein said antenna is dimensionally and flexibly configured and encased to wrap and conform to the sides and top of the tail covering transmission angles between minus 170 degrees and positive 170 degrees from tail top center vertical.

21. The health tracker device of claim 19, wherein said antenna is dimensionally configured to wrap substantially around the animal's tail to optimize transmission direction according to the diameter of the animal's tail.

22. The health tracker device of claim 19, wherein said antenna is created by printed undulating traces configured to optimize transmission strength and conformity according to the diameter of the animal's tail.

23. The health tracker device of claim 19, wherein said expandable band contains the flexible portion of a rigid-flex PCB where the inner layer of the flexible circuit portion includes a reflective layer below the antenna traces.

24. An application for monitoring health of an animal, including a home screen, onboarding module, health tracker device module, map module, devices module, and stable module stored on non-transitory computer readable media.

25. The application of claim 24, wherein said onboarding module allows a user to sign up on said application and create a username and password.

26. The application of claim 24, wherein said home screen allows the user to login with a username and password, and provides access to said onboarding module, said health tracker device module, said map module, said devices module, and said stable module.

27. The application of claim 24, wherein said health tracker device modules provides a list of monitors, allows access to details of each monitor, and allows for the creation of new monitors.

28. The application of claim 24, wherein said map module allows the user to view where animals being tracked are located.

29. The application of claim 24, wherein said devices module provides a list of all devices connected with said application.

30. The application of claim 24, wherein said stable modules allows a user to view all animals being tracked and allows the user to add an animal profile.

31. A method of using an application for monitoring the health of an animal, including the steps of:
a user onboarding with an onboarding module;
the user logging in on a home screen;
the user adding an animal profile in a stable module;
the user adding a monitor in a health tracker device module; and
the user viewing the location of animals being tracked with the map module.

32. The method of claim 31, further including the step of viewing details of a monitor in the health tracker device module.

33. The method of claim 31, further including the step of the user viewing devices linked with said application in a devices module.

34. A method of tracking the health of an animal, including the steps of:
securing a health tracker device to a tail of the animal, wherein the health tracker device includes an electronics and hardware portion operatively attached to an expandable band; and
detecting and tracking heart rate, temperature, GPS position, respiratory rate, blood oxygen levels, and movement of the animal with the health tracker device.

35. The method of claim 34, wherein the animal is chosen from the group consisting of horses, cows, sheep, pigs, dogs, cats, giraffes, rhinoceroses, camels, donkeys, alpacas, buffalo, and goats.

36. The method of claim 34, wherein said securing step is further defined as securing the expandable band including a securing mechanism to the tail.

37. The method of claim 34, wherein said securing step is further defined as securing the health tracker device at a base of the tail such that the electronics and hardware portion faces against a ventral face of the tail in contact with the animal's skin.

38. The method of claim 34, wherein said detecting and tracking step is further defined as continually detecting and tracking heart rate, temperature, GPS position, respiratory rate, blood oxygen levels, and movement.

39. The method of claim 34, further including the step of sending data detected and tracked to an application on a mobile device or a communications hub.

40. The method of claim 34, wherein said method is used for general health monitoring of the animal.

41. The method of claim 34, wherein said method is used while training the animal, and said detecting and tracking step is further defined as detecting and tracking heart rate, respiratory rate, blood oxygen, temperature, movement, GPS location, stride length, speed, balance/gait, force/impulsion/impact, and recovery/fitness data including heart rate return, heart rate variability, excess post-exercise oxygen consumption, and VO2 max.

42. The method of claim 41, further including the step of determining lameness of the animal.

43. The method of claim 41, further including the step of adjusting training of the animal based parameters detected.

44. The method of claim 34, wherein said method is used for monitoring foals and birth prediction, and further includes the step of detecting a change in heart rate, respiratory rate, pulse oxygen, temperature, and movement/activity to predict birth.

45. The method of claim 34, further including the steps of providing vibration to the animal to simulate a slow heart rate and calming the animal down.

46. The method of claim 34, further including the step of monitoring an animal with illness based on temperature and heart rate.

47. The method of claim 34, wherein the health tracker device remains on the animal for a period of time chosen from the group consisting of hours, days, weeks, and months.

48. The method of claim 34, further including the steps of detecting and tracking glucose, blood pressure, and electrolyte and hydration levels with the health tracker device.

49. The method of claim 34, further including the step of a rider viewing parameters detected by the health tracker device with a rider mobile device while riding the animal.

50. A method of tracking the health of an animal during training, including the steps of:
securing a health tracker device to the tail of the animal, wherein the health tracker device includes an electronics and hardware portion operatively attached to an expandable band;
detecting and tracking heart rate, respiratory rate, blood oxygen, temperature, movement, GPS location, stride length, speed, balance/gait, force/impulsion/impact, and recovery/fitness data including heart rate return, heart rate variability, excess post-exercise oxygen consumption, and VO2 max during a training session with the health tracker device; and
adjusting the training based on data detected.

51. A method of monitoring foals, including the steps of:
securing the health tracker device to the tail of a pregnant horse, wherein the health tracker device includes an electronics and hardware portion operatively attached to an expandable band;
detecting and tracking heart rate, respiratory rate, pulse oxygen, temperature, and movement/activity with the health tracker device; and
predicting birth of a foal in the horse.

52. A method of calming an animal, including the steps of:
securing the health tracker device to the tail of the animal, wherein the health tracker device includes an electronics and hardware portion operatively attached to an expandable band;
detecting and tracking heart rate, temperature, GPS position, respiratory rate, blood oxygen levels, and movement with the health tracker device; and
providing vibration to the animal and simulating a low-normal heart rate to calm the animal.
